(19) United States
(12) Patent Application Publication
(10) Pub. No.: US 2023/0000638 A1
(43) Pub. Date: Jan. 5, 2023

(54) CAGE FOR MINIMAL INVASIVE SURGERY

(71) Applicant: ENDAC CO.,LTD., Gyeonggi-do (KR)

(72) Inventors: Kyung Hak KIM, Gyeonggi-do (KR); Gil Sam LEE, Gyeonggi-do (KR)

(21) Appl. No.: 17/931,989

(22) Filed: Sep. 14, 2022

Related U.S. Application Data
(63) Continuation of application No. PCT/KR2021/003131, filed on Mar. 15, 2021.

(30) Foreign Application Priority Data
Mar. 18, 2020 (KR) 10-2020-0033336

(51) Int. Cl. A61F 2/44

(52) U.S. Cl. CPC A61F 2/442; A61F 2002/30593

(57) ABSTRACT

Disclosed is a cage for minimally invasive surgery, the cage including a main body part inserted into a space between a vertebra and an adjacent vertebra, and having lengthwise first and second end parts connected rotatably to each other, and a dilatation induction part provided in an inner space of the main body part, the dilatation induction part being configured to dilate a lengthwise middle part of the main body part by moving to a position at which the first and second end parts of the main body part are connected to each other.

### CROSS REFERENCE TO RELATED APPLICATION

This is a continuation of International Application No. PCT/KR2021/003131 filed on Mar. 15, 2021, which claims priority from Korean Patent Application No. 10-2020-0033336 filed on Mar. 18, 2020. The contents of these applications are incorporated herein by reference in their entireties.

### TECHNICAL FIELD

The present disclosure relates generally to a cage for minimally invasive surgery. More particularly, the present disclosure relates to a cage for minimally invasive surgery, which is configured to be inserted into a space between a vertebra and an adjacent vertebra so as to prevent subsidence thereof, to be rotated in the space between the vertebrae so as to increase the area of supporting the vertebrae, and to increase an interval between vertebral bodies which is narrowed.

### BACKGROUND

Generally, in the case of conventional laparotomy for treating a patient, the patient's recovery after surgery is slow due to a large incision site and a large amount of bleeding that occurs during surgery, and after surgery, a large scar remains, which affects the patient's life after the surgery.

In order to overcome the shortcomings of the laparoscopic surgery, a new surgical technique of minimally invasive surgery (MIS) in which a laparoscopic surgical instrument is used is being developed.

The minimally invasive surgery is a surgical technique in which only a minimal part is incised on a patient's body surface by using a long and thin surgical instrument specially designed to minimize an incised part required for surgery.

In the minimally invasive surgery, fewer incisions for a procedure are required, and the amount of bleeding during the procedure is significantly less than that of laparotomy, so a period for which a patient is recovered after the surgery is short and the patient has small external scars, so the number of such procedures has increased significantly in recent years.

Meanwhile, disc that exists between a vertebra and a bone functions as a joint and plays a very important role in minimizing impact applied to the vertebra as the position and shape of nucleus pulposus accommodated inside the disc changes according to the movement of the vertebra.

Most of the nucleus pulposus consists of water (water), but as a person ages, the amount of water gradually decreases and the disc loses its cushioning function.

Due to this, when excessive pressure is applied to fibers, back pain occurs, and when it progresses further, the fibers are severely stretched or ruptured and press nerve roots located behind the fibers, causing pain in the pelvis and legs.

After that, various side effects occur, such as an interval between the vertebrae gradually narrows or vertebral bones subside, resulting in spinal deformity.

As a method of treating diseases accompanied by damage to the disc, there is a method in which space between two adjacent vertebrae is replaced with a prosthetic material, which is a so-called cage, after the damaged intervertebral disc is removed. That is, the prosthetic material restores spinal function by restoring an original distance between two adjacent vertebral bodies, which is the original height of the intervertebral disc.

As a surgical method of inserting such a prosthetic material between vertebrae, there are anterior lumbar interbody fusion (ALIF), which inserts a prosthetic material from the front of the spine, lateral lumbar interbody fusion (LLIF), which inserts a prosthetic material through the flank, transforaminal lumbar interbody fusion (LIF), which inserts a prosthetic material in a diagonal direction at a distance of 30 to 40 mm from the center of the rear side of the back, and posterior lumbar interbody fusion (PLIF), which inserts a prosthetic material from the rear side of the back.

From the above point of view, “EXPANDABLE CAGE BETWEEN VERTEBRAL BODIES” (hereinafter, referred to as “Prior art 1”) is disclosed in Korean Patent No. 10-1352820.

Prior art 1 discloses an expandable cage between vertebral bodies which is inserted into a space between vertebral bodies so as to expand the space between vertebral bodies. The expandable cage includes: a cage body including an upper support part which is located at an upper part between the vertebral bodies so as to support the vertebral bodies, a lower support part which is located at a lower part between the vertebral bodies supports so as to support the vertebral bodies, and a connection part connecting the upper support part with the end of the lower support part; and a sliding member which increases or decreases an interval between the upper support part and the lower support part by sliding between the upper support part and the lower support part.

For reference, all bones including vertebrae are generally composed of the outermost cortical bone composed of dense and hard tissues, and the spongy bone composed of sparse tissues at the inner side of the cortical bone.

However, most of cages, including the cage of Prior art 1, do not support a part of the cortical bone, so as a long time passes after a procedure, subsidence occurs in which a cage is buried in the spongy bone.

In order to solve the above problem, Korean Patent No. 10-1730476 discloses “SPINAL IMPLANT WITH JOINT FOR MINIMALLY INVASIVE SURGERY” (hereinafter, referred to as “Prior art 2”).

In Prior art 2, a spinal implant includes: a body unit inserted into a space between a vertebra and an adjacent vertebra, first and second end parts of the body unit being rotatable to each other relative to a middle part between the first and second end parts; a rotation auxiliary unit which is formed on the middle part of the body unit and provides the reference point of the rotation of the first end part and the second end part to each other; and an operation unit inserted from the second end part of the body unit, the operation unit embodying the relative rotations of the first end part and the second end part to each other by being engaged with the rotation auxiliary unit.

However, the spinal implant of Prior art 2 has an advantage in that the spinal implant can rotate inside space between vertebrae to stably support upper and lower vertebral bodies, but a configuration required to perform the relative rotations of the first and second end parts of the body unit to each other are complicated, and a surgeon is required to use a separately designed surgical instrument so as to operate the operation unit of the body unit.

### BRIEF SUMMARY

The present disclosure has been made to solve the above problemsby providing a cage for minimally invasive surgery in which a pair of support bodies is rotated to each other inside space between vertebral bodies by a surgeon's simple manipulation so as to secure a sufficient support area, and is dilated even in a vertical direction so as to prevent subsidence between a vertebra and an adjacent vertebra.

A cage for minimally invasive surgery according to the present disclosure includes: a main body part inserted into a space between a vertebra and an adjacent vertebra, and having lengthwise first and second end parts connected rotatably to each other; and a dilatation induction part provided in an inner space of the main body part, the dilatation induction part being configured to dilate a lengthwise middle part of the main body part by moving to a position at which the first and second end parts of the main body part are connected to each other.

In addition, the main body part may be rectilinearly inserted into the space between the vertebra and the adjacent vertebra, and when a front end part of the main body part is in contact with a tissue disposed in the space, the first and second end parts of the main body part may be rotated to each other.

Furthermore, the main body part may include: a first support body whose first end part is initially inserted into a space between a vertebra and an adjacent vertebra, the first support body having a first space part formed therein; and a second support body whose first end part is connected rotatably to a second end part of the first support body, the second support body having a second space part communicable with the first space part formed therein.

Additionally, while the dilatation induction part is received in the second space part, the dilatation induction part may be moved to a position at which the first support body and the second support body are connected to each other, and may dilate the second end part of the first support body and the first end part of the second support body in upward and downward directions.

In addition, when the dilatation induction part is moved to the position at which the first support body and the second support body are connected to each other, an upper surface of the first support body may be disposed to be inclined upward from a first end of the first support body toward a second end thereof, and a lower surface of the first support body may be disposed to be inclined downward from the first end of the first support body toward the second end thereof, and an upper surface of the second support body may be disposed to be inclined downward from a first end of the second support body toward a second end thereof, and a lower surface of the second support body may be disposed to be inclined upward from the first end of the second support body toward the second end thereof.

Furthermore, the dilatation induction part may include: a moving block received in the second space part; and a connecting body provided on a second end part of the moving block, the connecting body being exposed to an outside at a second end part of the second support body and being connected to a surgical instrument.

Additionally, the connecting body may include: an insertion member inserted into an insertion hole famed in the second end part of the moving block; and a coupling member connected with the insertion member and having a diameter larger than a diameter of the insertion member, the coupling member being exposed to the outside at the second end part of the second support body and being coupled to a front end part of the surgical instrument.

In addition, a coupling groove into which the front end part of the surgical instrument is inserted may be formed in a second surface of the coupling member.

Furthermore, a threaded part may be formed on an outer circumferential surface of the coupling member.

Additionally, a guide hole through which the coupling member passes may be formed in the second end part of the second support body, and a threaded part may be formed on an inner circumferential surface of the second end part of the second support body which partitions the guide hole such that the threaded part formed on the inner circumferential surface is coupled to the threaded part formed on the outer circumferential surface of the coupling member.

In addition, a gripping groove gripped by the surgical instrument may be formed in the second end part of the second support body.

Furthermore, the cage may further include: a rotating part provided at a position at which the first support body and the second support body are connected to each other, wherein the rotating part may include: a pair of rotating plates; a rotating pin foiled on each of the rotating plates; and a through hole through which the rotating pin is rotatably inserted.

Additionally, a distance between the pair of rotating plates spaced apart from each other may be smaller than a thickness of the moving block.

In addition, the cage may further include: a movement prevention means provided on a surface of the lengthwise second end part of the first support body and a surface of the lengthwise first end part of the second support body which are in contact with each other by facing each other, wherein the movement prevention means may be configured to have a sawtooth shape in which ridges and grooves are continuously formed, the movement prevention means being arranged circumferentially around the rotating pin and circumferentially around the through hole.

The cage for minimally invasive surgery according to the present disclosure is provided with the main body part configured such that the lengthwise first and second end parts of the main body part are rotatable while the main body part is rectilinearly inserted into a space between a vertebra and an adjacent vertebra, thereby increasing the area of supporting vertebrae so as to effectively prevent subsidence thereof.

In addition, due to the dilatation induction part which slides inside the main body part and dilates the lengthwise middle part of the main body part in upward and downward directions, the cage for minimally invasive surgery according to the present disclosure can support vertebrae more stably and can flexibly respond to weight, impact, and torsional stress caused by various movements, which are applied to a vertebra of a person which undergoes a surgical procedure.

Furthermore, the cage for minimally invasive surgery according to the present disclosure has a simple structure in which the lengthwise first and second end parts of the main body part are rotatable to each other and the lengthwise middle part of the main body part is dilatable, thereby increasing a surgeon's surgical convenience and improving a surgical success rate.

### BRIEF DESCRIPTION OF DRAWINGS

FIG. 1 is a perspective view of a cage for minimally invasive surgery according to an embodiment of the present disclosure.

FIG. 2 is a side view of the cage for minimally invasive surgery according to the embodiment of the present disclosure.

FIG. 3 is a top plan view of the cage for minimally invasive surgery according to the embodiment of the present disclosure.

FIG. 4 is a perspective view illustrating the rotated state of the cage for minimally invasive surgery according to the embodiment of the present disclosure.

FIG. 5 is a top plan view of the cage for minimally invasive surgery illustrated in FIG. 4.

FIG. 6 is a perspective view illustrating a state in which the cage for minimally invasive surgery according to the embodiment of the present disclosure is dilated by the movement of a dilatation induction part.

FIG. 7 is a perspective view illustrating the configuration of the dilatation induction part according to the embodiment of the present disclosure.

FIG. 8 is an exploded perspective view of the dilatation induction part illustrated in FIG. 7.

FIG. 9 is a cross-sectional view of the dilatation induction part illustrated in FIG. 7.

FIG. 10 is a side view illustrating movement prevention means according to the embodiment of the present disclosure.

FIG. 11 is an exploded perspective view of a first support body and a second support body provided respectively with the movement prevention means of FIG. 10.

FIG. 12 is a cross-sectional view of a dilatation induction part according to another embodiment of the present disclosure.

FIG. 13 is an exploded perspective view of the dilatation induction part according to the another embodiment of the present disclosure.

### DETAILED DESCRIPTION OF EMBODIMENTS

Advantages and features of the present disclosure and methods of achieving them will become apparent with reference to embodiments described below in detail in conjunction with the accompanying drawings.

However, the present disclosure is not limited to the embodiments disclosed below, but will be embodied in various different forms. These embodiments only allow the present disclosure to be complete and are provided to fully inform the scope of the present disclosure to those skilled in the art to which the present disclosure pertains, and the present disclosure is only defined by the scope of the claims.

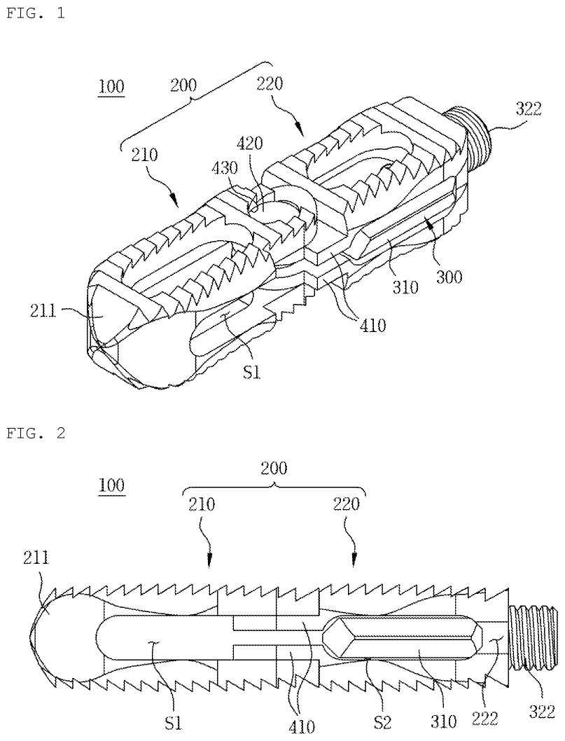

Hereinafter, a cage for minimally invasive surgery according to the embodiment of the present disclosure will be described in detail with reference to FIGS. 1 to 13. In describing the present disclosure, detailed descriptions of related well-known functions or configurations are omitted so as not to obscure the gist of the present disclosure.

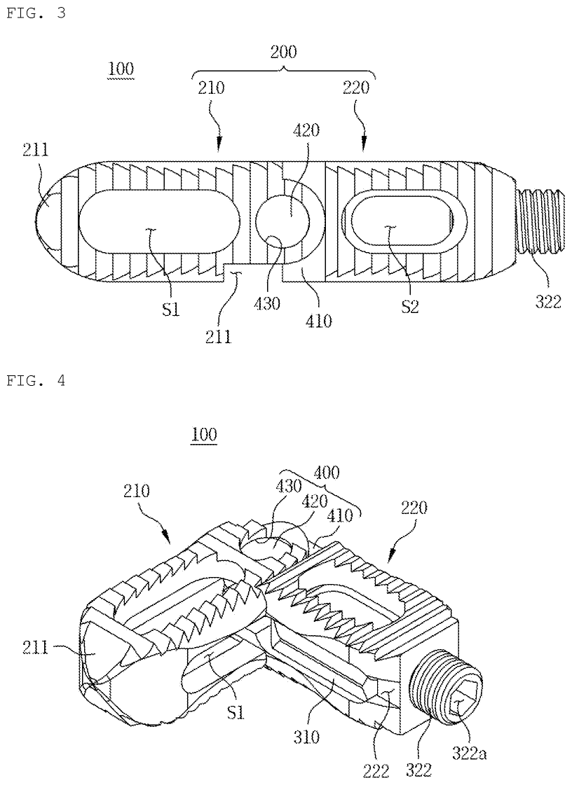

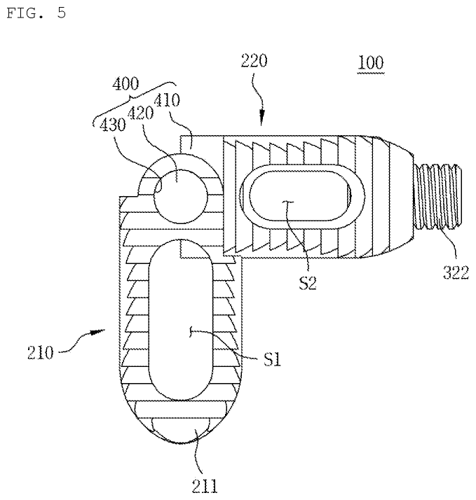

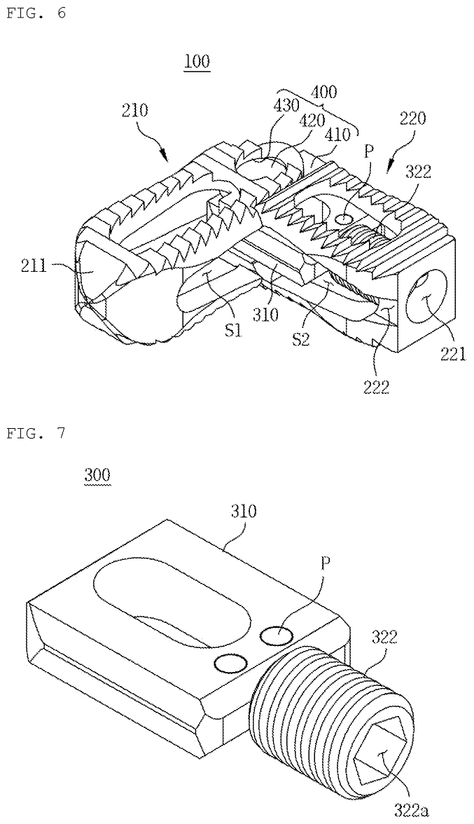

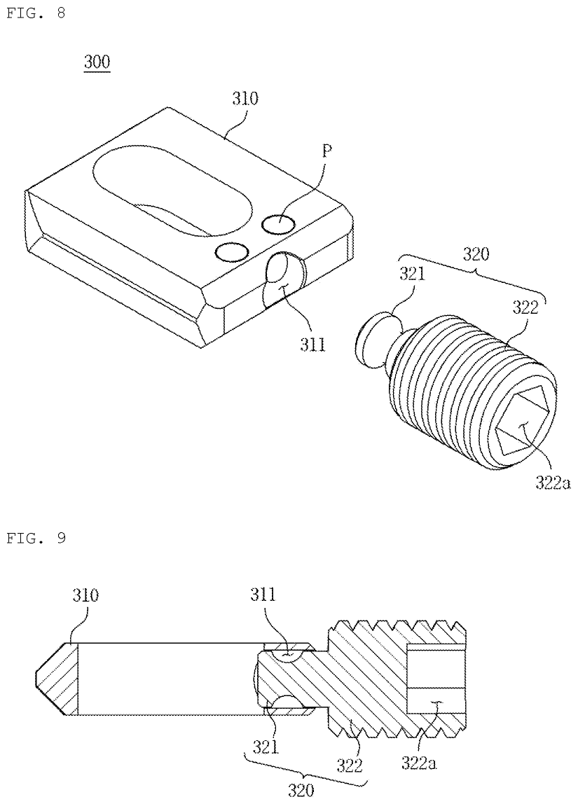

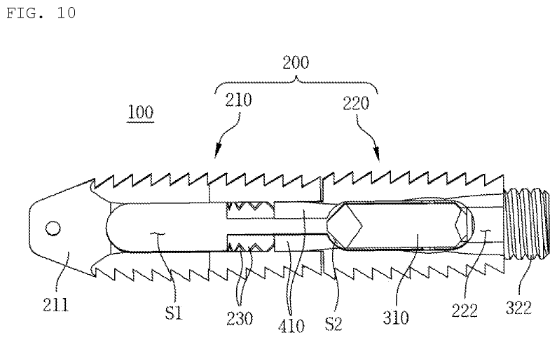

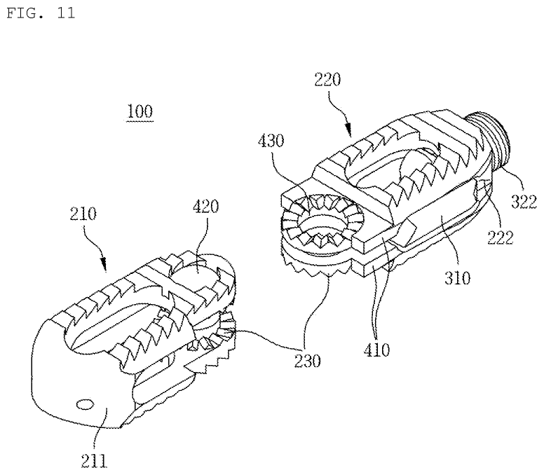

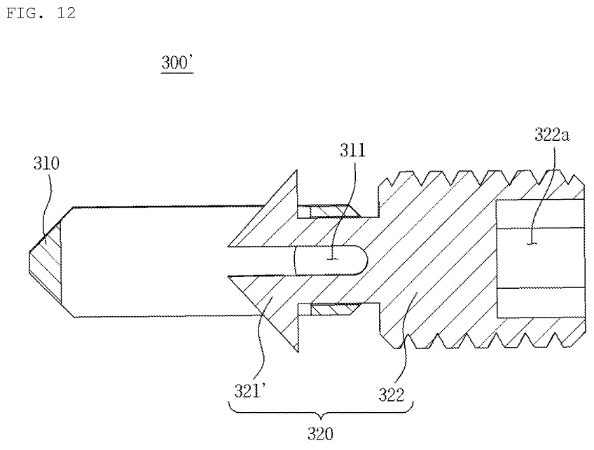

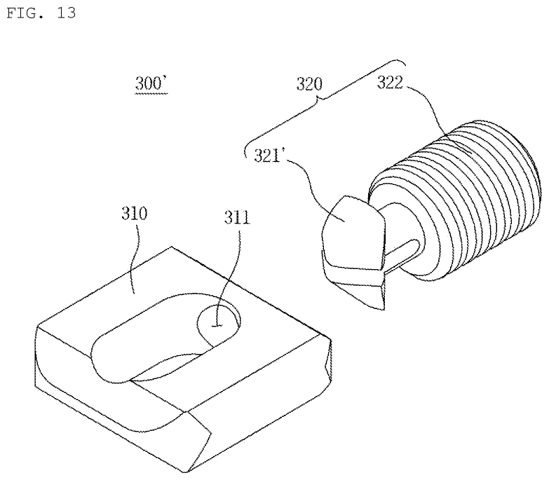

FIG. 1 is a perspective view of a cage for minimally invasive surgery according to an embodiment of the present disclosure, FIG. 2 is a side view of the cage for minimally invasive surgery according to the embodiment of the present disclosure, FIG. 3 is a top plan view of the cage for minimally invasive surgery according to the embodiment of the present disclosure, FIG. 4 is a perspective view illustrating the rotated state of the cage for minimally invasive surgery according to the embodiment of the present disclosure, FIG. 5 is a top plan view of the cage for minimally invasive surgery illustrated in FIG. 4, FIG. 6 is a perspective view illustrating a state in which the cage for minimally invasive surgery according to the embodiment of the present disclosure is dilated by the movement of a dilatation induction part, FIG. 7 is a perspective view illustrating the configuration of the dilatation induction part according to the embodiment of the present disclosure, FIG. 8 is an exploded perspective view of the dilatation induction part illustrated in FIG. 7, FIG. 9 is a cross-sectional view of the dilatation induction part illustrated in FIG. 7, FIG. 10 is a side view illustrating movement prevention means according to the embodiment of the present disclosure, FIG. 11 is an exploded perspective view of a first support body and a second support body provided respectively with the movement prevention means of FIG. 10, FIG. 12 is a cross-sectional view of a dilatation induction part according to another embodiment of the present disclosure, and FIG. 13 is an exploded perspective view of the dilatation induction part according to the another embodiment of the present disclosure.

As illustrated in FIGS. 1 to 9, the cage 100 for minimally invasive surgery according to the embodiment of the present disclosure may include a main body part 200 inserted into a space between a vertebra and an adjacent vertebra, and having lengthwise first and second end parts connected rotatably to each other; and the dilatation induction part 300 provided in the inner space of the main body part 200, the dilatation induction part 300 being configured to dilate the lengthwise middle part of the main body part 200 by moving to a position at which the first and second end parts of the main body part 200 are connected to each other.

The main body part 200 may be configured to be rectilinearly inserted into a space between a vertebra and an adjacent vertebra and to be rotated to have an approximately “L” shape in the space so as to increase an area to support the vertebrae, and the dilatation induction part 30 may be configured to allow the main body part 200 transformed to have the “L” shape to be dilated such that an interval between vertebral bodies which is narrowed is increased.

First, the main body part 200 may be rectilinearly inserted into a space between a vertebra and an adjacent vertebra, and when the front end part of the main body part is in contact with a tissue disposed in the space, the lengthwise first and second end parts of the main body part may be rotated to each other.

As illustrated in FIGS. 1 to 3, the main body part 200 may include the first support body 210 whose lengthwise first end part is initially inserted into a space between a vertebra and an adjacent vertebra, the first support body 210 having a first space part S1 formed therein; and the second support body 220 whose lengthwise first end part is connected rotatably to the lengthwise second end part of the first support body 210, the second support body 220 having a second space part S2 communicable with the first space part S1 formed therein.

The first support body 210 may have a “U”-shaped sectional shape as a whole, and an opening part communicable with the first space part S1 described above may be formed in each of the upper and lower surfaces of the first support body 210.

In addition, a cap 211 may be formed on the lengthwise first end part of the first support body 210. The cap 211 may function to allow the first support body 210 to be easily inserted into a space between vertebral bodies, and may be formed as a curved surface having a round shape so as to prevent damage to a tissue and to facilitate the insertion of the first support body 210.

The second support body 220 may also have a “U”-shaped sectional shape as a whole, and an opening part communicable with the second space part S2 described above may be formed in each of the upper and lower surfaces of the second support body 220. Additionally, a guide hole 221 may be formed in the lengthwise second end part of the second support body 220 such that a coupling member 322 of the dilatation induction part 300 to be described later can pass through the guide hole 221 (see FIG. 6).

Meanwhile, multiple serrated protrusions may be formed respectively on the upper and lower surfaces of each of the first support body 210 and the second support body 220. These protrusions function to prevent the first support body 210 and the second support body 220 from being moved between vertebral bodies. That is, the protrusions may be considered to assist the seating of the first support body 210 and the second support body 220 between vertebral bodies.

While the dilatation induction part 300 is received in the second space part S2 of the second support body 220, the dilatation induction part 300 is moved to a position at which the first support body 210 and the second support body 220 are connected to each other, and dilates the lengthwise second end part of the first support body 210 and the lengthwise first end part of the second support body 220 in upward and downward directions.

As illustrated in FIGS. 7 to 9, the dilatation induction part 300 may include a moving block 310 received in the second space part S2 formed in the second support body 220; and a connecting body 320 provided on the lengthwise second end part of the moving block 310, the connecting body 320 being exposed to the outside at the lengthwise second end part of the second support body 220 and being connected to a surgical instrument.

The moving block 310, which has a rectangular shape as a whole, may be received in the second space part S2, and the front end part of the moving block 310 and the other end part thereof may be processed to be round or chamfered and may have such that the moving block 310 is easily inserted into a position between a pair of rotating plates 410 to be described later. Additionally, a through hole may be formed in the center portion of the moving block 310 such that the through hole is communicable with the opening parts formed in the upper and lower surfaces of the second support body 220.

The connecting body 320 may include: an insertion member 321 inserted into an insertion hole 311 formed in the lengthwise second end part of the moving block 310; and the coupling member 322 connected with the insertion member 321 and having a diameter larger than the diameter of the insertion member 321, the coupling member being exposed to the outside at the lengthwise second end part of the second support body 220 and being coupled to the front end part of a surgical instrument.

The insertion member 321 may be inserted into the insertion hole 311 of the moving block 310 by a forcible fitting method such that the insertion member 321 can be fixedly connected to the moving block 310. Furthermore, in the insertion hole 311, the insertion member 321 may be maintained to be fixed to the moving block 310 of the moving block 310 by a fixing pin P.

When applying the fixing pin P to fix the insertion member 321 to the moving block 310, as illustrated in FIG. 8, pin holes into which a pair of fixing pins P can be inserted may be formed in the moving block 310 such that the pin holes are communicable with the insertion hole 311 of the moving block 310. Additionally, the insertion member 321 may have a lengthwise middle part having a bottleneck shape such that the movement of the insertion member 321 is prevented by the pair of fixing pins P. That is, as illustrated in FIG. 8, the outer diameter of the lengthwise middle part of the insertion member 321 may be smaller than the outer diameters of the lengthwise first and second end parts of the insertion member 321.

Accordingly, when the fixing pins P are inserted into the pin holes in a state in which the insertion member 321 is inserted into the insertion hole 311 of the moving block 310, the pair of pin holes may pass by the lengthwise middle part of the insertion member 321 perpendicularly thereto and may be disposed between the lengthwise first and second end parts of the insertion member 321. In this case, the lengthwise first or second end part of the insertion member 321 may be prevented from moving forward or rearward by the pair of fixing pins P, so the connecting body 320 may be securely coupled to the lengthwise second end part of the moving block 310.

The coupling member 322 may be connected integrally with the lengthwise second end part of the insertion member 321, and has an outer diameter so as not to pass through the insertion hole 311 of the moving block 310.

In addition, the lengthwise first end part of the coupling member 322 may be inserted into the guide hole 221 formed in the lengthwise second end part of the second support body 220, and the remaining lengthwise portion thereof may be disposed to be exposed to the outside of the second support body 220. Additionally, a coupling groove 322a into which the front end part of the surgical instrument can be inserted may be formed in the lengthwise second end part of the coupling member 322.

For reference, in the embodiment of the present disclosure, the coupling groove 322a is illustrated to have the shape of a groove having a hexagonal cross-section in the drawing, but is not limited thereto. That is, the coupling groove 322a may be formed in the lengthwise second end part of the coupling member 322 by having various shapes corresponding to the front end part of the surgical instrument.

Meanwhile, the guide hole 221 formed in the lengthwise second end part of the second support body 220 determines the position of the coupling member 322 such that the moving block 310 is stably disposed in the second space part S2 of the second support body 220 and, further, allows the moving block 310 to reciprocate only in a rectilinear direction.

That is, the guide hole 221 prevents the moving block 310 from being disposed at one side in the second space part S2, and prevents the moving block 310 from being exposed to the widthwise first or second side of the second support body 220 as illustrated in FIG. 3. Accordingly, the moving block 310 may move rectilinearly in a preset direction. Here, the preset direction may be a direction in which the lengthwise first end part of the moving block 310 moves toward a position at which the first support body 210 is connected with the second support body 220.

In addition, the guide hole 221 may prevent the moving block 310 from being moved inside the second space part S2 by an unexpected external force.

In order to further increase the effects obtained by the guide hole 221 as described above, threaded parts which can be screwed to each other are preferably formed respectively on the outer circumferential surface of the coupling member 322 and the inner circumferential surface of the lengthwise second end part of the second support body 220 which partitions the guide hole 221.

In this case, as illustrated in FIG. 6, when the lengthwise first end part of the moving block 310 is moved to the connection position of the first support body 210 and the second support body 220 to each other, that is, to a position at which a rotating part 400 to be described later is disposed, the coupling member 322 may have a length to be received in the second space part S2 of the second support body 220 through the guide hole 221.

In other words, the coupling member 322 preferably has a length to be screwed to the lengthwise second end part of the second support body 220 only until the lengthwise first end part of the moving block 310 is moved to the connection position of the first support body 210 and the second support body 220 to each other so as to dilate the lengthwise second end part of the first support body 210 and the lengthwise first end part of the second support body 220 in a vertical direction.

This is intended for a surgeon to intuitively recognize the dilation of the first support body 210 and the second support body 220 due to the movement of the lengthwise first end part of the moving block 310 to the connection position of the first support body 210 and the second support body 220 to each other, and further, is intended to prevent a tissue between vertebral bodies from being damaged or pressurized when the lengthwise first end part of the moving block 310 protrudes from the first support body 210 or the second support body 220 since the lengthwise first end part of the moving block 310 is moved beyond a preset position due to a surgeon's mistake.

Accordingly, as illustrated in FIG. 3, after the main body part 200 composed of the first support body 210 and the second support body 220 is rectilinearly inserted into a space between a vertebra and an adjacent vertebra, the main body part 200 may be transformed into an approximately “L” shape, and in this transformed state, the moving block 310 of the dilatation induction part 300 may be moved toward the connection position of the first support body 210 and the second support body 220 to each other from the second space part S2 of the second support body 220.

That is, when a surgeon rotates the coupling member 322 of the dilatation induction part 300 by using a surgical instrument, the moving block 310 may be moved to the connection position of the first support body 210 and the second support body 220 to each other while the coupling member 322 is inserted into the second space part S2 of the second support body 220 as illustrated in FIG. 6.

In this state, the upper surface of the first support body 210 may be disposed to be inclined upward from the lengthwise first end of the first support body 210 toward the lengthwise second end thereof, and the lower surface of the first support body 210 may be disposed to be inclined downward from the lengthwise first end of the first support body 210 toward the lengthwise second end thereof.

Furthermore, the upper surface of the second support body 220 may be disposed to be inclined downward from the lengthwise first end of the second support body 220 toward the lengthwise second end thereof, and the lower surface of the second support body 220 may be disposed to be inclined upward from the lengthwise first end of the second support body 220 toward the lengthwise second end thereof.

As described above, since the lengthwise second end part of the first support body 210 and the lengthwise first end part of the second support body 220 are dilated in upward and downward directions, the main body part can more stably support a vertebra and an adjacent vertebra in space therebetween and can flexibly respond to weight, impact, and torsional stress caused by various movements, which are applied to the vertebrae of a person which undergoes a surgical procedure.

For reference, as illustrated in FIGS. 2, 4, and 6, an engraved gripping groove 222 may be formed in each of the opposite side surfaces of the lengthwise second end part of the second support body 220 such that the gripping groove can be gripped by a surgical instrument. Additionally, as illustrated in FIG. 3, a cut part 211 may be formed in the upper surface of the lengthwise second end part of the first support body 210. During the rotation of the first support body 210, the cut part 211 prevents the lengthwise second end part of the first support body from interfering with the lengthwise first end part of the second support body 220, and may be formed in the rotating direction of the first support body 210.

Meanwhile, the cage 100 for minimally invasive surgery according to the embodiment of the present disclosure may include the rotating part 400 provided in the lengthwise middle point of the main body part 200.

The rotating part 400 may allow the lengthwise second end part of the first support body 210 and the lengthwise first end part of the second support body 220 to be connected rotatably to each other, and as illustrated in FIGS. 1 and 2, may include the pair of rotating plates 410, a rotating pin 420 formed in each of the rotating plates 410, and a through hole 430 into which the rotating pin 420 is rotatably inserted.

The rotating plates 410 may be selectively provided on the lengthwise second end part of the first support body 210 or the lengthwise first end part of the second support body 220. In the embodiment of the present disclosure, as illustrated in FIGS. 1 to 3, the rotating plates 410 are illustrated to be provided on the lengthwise first end part of the second support body 220.

In other words, as illustrated in FIG. 2, in any one of the pair of rotating plates 410, a lengthwise second end thereof is connected integrally with a ceiling surface which partitions the second space part S2 of the second support body 220 and a lengthwise first end thereof extends toward the first space part S1 of the first support body 210, and in the remaining one of the rotating plates 410, a lengthwise second end thereof is connected to a bottom surface which partitions the second space part S2 of the second support body 220 and a lengthwise first end thereof extends toward the first space part S1 of the first support body 210. In this case, the pair of rotating plates 410 may be disposed to be spaced apart from each other by having a predetermined interval in a vertical direction.

The rotating pin 420 may be provided on each of the pair of rotating plates 410. That is, the rotating pins 420 may be respectively provided on the upper surface of the rotating plate 410 connected with the ceiling surface of the second support body 220 and the lower surface of the rotating plate 410 connected with the bottom surface of the second support body 220.

In addition, the through hole 430 may be formed in each of the upper and lower surfaces of the lengthwise second end part of the first support body 210.

The first support body 210 and the second support body 220 may be connected rotatably to each other by the rotating part 400 having the above configuration. Meanwhile, a distance between the pair of rotating plates 410 spaced apart from each other may be smaller than the thickness of the moving block 310. This is because the pair of rotating plates 410 are required to have an increased vertical distance therebetween by being pressurized by the moving block 310 such that the lengthwise second end part of the first support body 210 and the lengthwise first end part of the second support body 220 are dilated in upward and downward directions by the movement of the moving block 310.

In addition, it is preferable that the lengthwise second end part of each of the pair of rotating plates 410 facing the lengthwise first end part of the moving block 310 has a curved surface by being processed to be round such that the moving block 310 can be easily inserted into a space between the pair of rotating plates 410.

Meanwhile, the cage 100 for minimally invasive surgery according to the embodiment of the present disclosure may further include the movement prevention means 230 which securely couples the lengthwise second end part of the first support body 210 to the lengthwise first end part of the second support body 220, and fixedly maintains the states of the first support body 210 and the second support body 220 rotated to each other.

As illustrated in FIGS. 10 and 11, the movement prevention means 230 is provided at a position at which the first support body 210 and the second support body 220 are connected to each other, and precisely, may be provided on each of the surfaces of the lengthwise second end part of the first support body 210 and the lengthwise first end part of the second support body 220 which are in contact with each other by facing each other.

As illustrated in FIGS. 10 and 11, the movement prevention means 230 may be configured to have a sawtooth shape in which ridges and grooves are continuously formed.

A movement prevention means 230 provided on the lengthwise second end part of the first support body 210 may be arranged circumferentially around the rotating pin 420, and a movement prevention means 230 provided on the lengthwise first end part of the second support body 220 may be arranged circumferentially around the through hole 430.

Accordingly, the movement prevention means 230 provided on the lengthwise second end part of the first support body 210 and the movement prevention means 230 provided on the lengthwise first end part of the second support body 220 may be coupled to each other by being engaged with each other. Particularly, when the first support body 210 and the second support body 220 are relatively rotated to each other, the first support body 210 and the second support body 220 may be prevented from being rotated in directions opposite to rotating directions thereof due to the coupling force of the movement prevention means 230.

Meanwhile, in FIGS. 12 and 13, a dilatation induction part 300′ according to the another embodiment of the present disclosure is illustrated.

The dilatation induction part 300′ according to the another embodiment of the present disclosure is provided with an insertion member 321′ having a structure different from the insertion member 321 of the dilatation induction part 300 according to the embodiment of the present disclosure, and remaining components according to the another embodiment may be considered the same.

Accordingly, hereinafter, only the insertion member 321′ will be described.

As illustrated in FIGS. 12 and 13, the insertion member 321′ has the shape of a hollow wedge as a whole, and may be made of a material having elastic restoring force such that the insertion member 321′ can be elastically closed by an external force and restored to an initial state thereof when the external force is released.

The front end part of the insertion member 321′ may be manufactured in the shape of an arrowhead having a size larger than the insertion hole 311 of the moving block 310. Accordingly, when the insertion member 321′ is inserted into the insertion hole 311, the front end part may be closed, and after the front end part passes through the insertion hole 311, the front end part may be opened and be restored to an initial state thereof.

In this case, the front end part of the insertion member 321′ is disposed in an opened state thereof in the inner space of the moving block 310 and performs a function like the function of a stopper. That is, the insertion member 321′ is in contact with the inner surface of the moving block 310 in a state in which the insertion member 321′ is opened so as not to pass through the insertion hole 311, and accordingly, the moving block 310 and the connecting body 320 may be coupled to each other such that the moving block 310 and the connecting body 320 are not separated from each other.

Particularly, the insertion member 321′ according to another embodiment of the present disclosure enables a worker to couple the connecting body 320 and the moving block 310 to each other in a one-touch method even without using a component such as the fixing pin P described in the embodiment of the present disclosure.

Although the specific embodiments according to the present disclosure have been described so far, the embodiments may be variously modified without departing from the scope of the present disclosure.

For example, the pair of rotating plates 410 of the rotating part 400 is formed integrally with the second support body 220, and correspondingly, the through hole 430 is formed in the first support body 210. This is described in the specification of the present disclosure and is illustrated in the drawing, but is not limited thereto. That is, the pair of rotating plates 410 may be provided integrally with the lengthwise second end part of the first support body 210, and correspondingly, the through hole 430 may be formed in the lengthwise first end part of the second support body 220.

However, in order to easily move the moving block 310 received in the second space part S2 of the second support body 220 to the connection position of the first support body 210 and the second support body 220 to each other, it is preferable that the pair of rotating plates 410 is provided integrally with the lengthwise first end part of the second support body 220, and correspondingly, the through hole 430 is formed in the lengthwise second end part of the first support body 210. This is because when the pair of rotating plates 410 is provided on the first support body 210, the moving block 310 interferes with a free end of each of the rotating plates 410 during the movement of the moving block 310, so the moving block 310 does not naturally move. Accordingly, it is preferable that the pair of rotating plates 410 of the rotating part 400 is provided integrally with the lengthwise first end part of the second support body 22, and correspondingly, the through hole 430 is formed in the lengthwise second end part of the first support body 210.

Therefore, the scope of the present disclosure should not be limited to the described embodiments, but should be defined by the claims to be described later and equivalents thereto.

The cage for minimally invasive surgery of the present disclosure may be applied to and sold in the medical industry.

1. A cage for minimally invasive surgery, the cage comprising:
a main body part inserted into a space between a vertebra and an adjacent vertebra, and having lengthwise first and second end parts connected rotatably to each other; and
a dilatation induction part provided in an inner space of the main body part, the dilatation induction part being configured to dilate a lengthwise middle part of the main body part by moving to a position at which the first and second end parts of the main body part are connected to each other.

2. The cage of claim 1, wherein the main body part is rectilinearly inserted into the space between the vertebra and the adjacent vertebra, and when a front end part of the main body part is in contact with a tissue disposed in the space, the first and second end parts of the main body part are rotated to each other.

3. The cage of claim 2, wherein the main body part comprises:
a first support body whose first end part is initially inserted into the space between the vertebra and the adjacent vertebra, the first support body having a first space part formed therein; and
a second support body whose first end part is connected rotatably to a second end part of the first support body, the second support body having a second space part communicable with the first space part formed therein.

4. The cage of claim 3, wherein while the dilatation induction part is received in the second space part, the dilatation induction part is moved to a position at which the first support body and the second support body are connected to each other, and dilates the second end part of the first support body and the first end part of the second support body in upward and downward directions.

5. The cage of claim 4, wherein when the dilatation induction part is moved to the position at which the first support body and the second support body are connected to each other, an upper surface of the first support body is disposed to be inclined upward from a first end of the first support body toward a second end thereof, and a lower surface of the first support body is disposed to be inclined downward from the first end of the first support body toward the second end thereof, and an upper surface of the second support body is disposed to be inclined downward from a first end of the second support body toward a second end thereof, and a lower surface of the second support body is disposed to be inclined upward from the first end of the second support body toward the second end thereof.

6. The cage of claim 3, wherein the dilatation induction part comprises:
a moving block received in the second space part; and
a connecting body provided on a second end part of the moving block, the connecting body being exposed to an outside at a second end part of the second support body and being connected to a surgical instrument.

7. The cage of claim 6, wherein the connecting body comprises:
an insertion member inserted into an insertion hole formed in the second end part of the moving block; and
a coupling member connected with the insertion member and having a diameter larger than a diameter of the insertion member, the coupling member being exposed to the outside at the second end part of the second support body and being coupled to a front end part of the surgical instrument.

8. The cage of claim 7, wherein a coupling groove into which the front end part of the surgical instrument is inserted is famed in a second surface of the coupling member.

9. The cage of claim 7, wherein a threaded part is foamed on an outer circumferential surface of the coupling member.

10. The cage of claim 9, wherein a guide hole through which the coupling member passes is formed in the second end part of the second support body, and a threaded part is formed on an inner circumferential surface of the second end part of the second support body which partitions the guide hole such that the threaded part formed on the inner circumferential surface is coupled to the threaded part formed on the outer circumferential surface of the coupling member.

11. The cage of claim 10, wherein a gripping groove gripped by the surgical instrument is formed in the second end part of the second support body.

12. The cage of claim 6, further comprising:
a rotating part provided at a position at which the first support body and the second support body are connected to each other,
wherein the rotating part comprises:
a pair of rotating plates;
a rotating pin formed on each of the rotating plates; and
a through hole through which the rotating pin is rotatably inserted.

13. The cage of claim 12, wherein a distance between the pair of rotating plates spaced apart from each other is smaller than a thickness of the moving block.

14. The cage of claim 12, further comprising:
a movement prevention means provided on a surface of the lengthwise second end part of the first support body and a surface of the lengthwise first end part of the second support body which are in contact with each other by facing each other,
wherein the movement prevention means is configured to have a sawtooth shape in which ridges and grooves are continuously formed, the movement prevention means being arranged circumferentially around the rotating pin and circumferentially around the through hole.
